(19) United States
(12) Patent Application Publication
(10) Pub. No.: US 2016/0333310 A1
(43) Pub. Date: Nov. 17, 2016

(54) CAPSULES CONTAINING CELLS WITH HEMATOPOIETIC POTENTIAL

(71) Applicants: ETABLISSEMENT FRANÇAIS DU SANG, La Plaine Saint Denis (FR); UNIVERSITE PIERRE ETMARIE CURIE (PARIS 6), Paris (FR); ECOLE SUPÉRIEURE DE PHYSIQUE ET DE CHIMIE INDUSTRIELLES DE LA VILLE DE PARIS, Paris (FR); ASSISTANCE PUBLIQUE HOPITAUX DE PARIS, Paris (FR); CENTRE NATIONAL DE LA RECHERCHE SCIENTIFIQUE (C.N.R.S.), Paris (FR)

(72) Inventors: Maria Catalina GIARRATANA, SAINT OUEN (FR); Luc DOUAY, PARIS (FR); Hugo DOMEJEAN, Saint Marcel (FR); Nicolas BREMOND, PARIS (FR); Julie Victoire BROUCHON, PARIS (FR); Jean BAUDRY, PARIS (FR); Jérôme BIBETTE, PARIS (FR)

(21) Appl. No.: 15/103,655

(22) PCT Filed: Dec. 12, 2014
(86) PCT No.: PCT/EP2014/077607

§ 371 Date: Jun. 10, 2016

(30) Foreign Application Priority Data
Dec. 13, 2013 (FR) 1362595

(51) Int. Cl. C12N 5/00; C12N 5/078

(52) U.S. Cl. CPC C12N 5/0012; C12N 5/0641; C12N 2501/14; C12N 2501/125; C12N 2501/2303; C12N 2501/145; C12N 2501/26; C12N 2501/155; C12N 2501/165; C12N 2501/2306

(57) ABSTRACT

The present invention relates to a capsule comprising at least one cell with hematopoietic potential, said capsule being formed with a liquid core and at least one gelled shell encapsulating totally the liquid core at its periphery, to the use of such a capsule for producing ex vivo enucleated erythroid cells as well as an ex vivo method for producing enucleated erythroid cells using said capsule.

The present invention relates to a capsule comprising at least one cell with hematopoietic potential, said capsule being formed with a liquid core and at least one gelled shell encapsulating totally the liquid core at its periphery, to the use of such a capsule for producing ex vivo enucleated erythroid cells as well as an ex vivo method for producing enucleated erythroid cells using said capsule.

There still exists a high demand for labile blood products, notably for transfusion purposes, and this demand is not satisfied by present supplies of natural human blood satisfactorily. Accordingly, many blood substitutes to natural blood have been studied.

However, recombinant or stabilized hemoglobins have shown disappointing performances, the indications of artificial oxygen carriers are limited and the development of “universal” red corpuscles compatible with the ABO system and/or with the RhD antigen by enzymatic treatment or antigenic masking is slow.

Therefore there exists a need for alternatives to these methods. In this respect, the attempts for producing erythroid cells such as red corpuscles from stem cells in vitro, are particularly encouraged.

However, it is a considerable challenge to reproduce in vitro which nature needs several months for building in vivo. During its development in humans, erythropoiesis changes from the mesodermis into two waves. Primitive erythropoiesis begins as soon as the third pregnancy week in the yolk sac (extra-embryonic tissue) and gives rise to megaloblastic nucleated primitive erythrocytes which synthesize embryonic hemoglobin of the Gower type I (ζ2ε2) and Gower II (α2ε2). Definitive erythropoiesis begins during the fifth pregnancy week in the region of the mesonephros aorta-gonads (AGA), before migrating towards the fetal liver and the bone marrow. Mature erythroid cells produce gradually, leading to the production of normocytic enucleated red corpuscles (RBC), and containing fetal hemoglobin (α2γ2) and then adult hemoglobin (α2β2).

To this day, several attempts for producing red corpuscles from human embryonic stems cells have been reported, as described by Ma. et al., (2008), Proc. Natl. Acad. Sci. USA, 105:13087-13092. However, these experiments are generally based on a co-culture step in the presence of stromal cells, which makes the intensification of the processes difficult.

For example mention may be made of patent application WO2005118780 which describes an in vitro method for massive and selective production of enucleated erythrocytes. According to this method, hematopoietic stem cells are cultivated in a culture medium which comprises at least one hematopoietic growth factor and then the culture of the cells, thereby obtained, is put into contact with the support cells.

Patent application WO2011101468 as for it describes a cell culture medium, without the requirement of an eco-culture on a cell stroma, for growth and/or differentiation of the cells of the hematopoietic line which comprises insulin, transferrin and plasma or serum.

The method for producing at an industrial scale of choice is today the reactor with stirring for example used for producing vaccines or monoclonal antibodies, with growth of the cells in solution or on microparticles for the adherent cells.

This production method nevertheless poses problems as soon as the cultures are made at significant cell densities, which is required for industrial production. Indeed, the greater the density of cells, the more the flows have to be increased and accordingly stirred. However, unlike yeasts and bacteria, mammal cells, notably stem cells are fragile and resist poorly to mechanical treatments, leading to much lower yields than expected.

Beads filled with alginate are used in a cell culture as a support for adherent cells wherein the cells are therefore located outside the beads.

Subsequently, the idea of encapsulating cells inside beads full of alginate was tackled. A first toxicity problem then appeared in the encapsulation process and many developments on the material and the gelling process were proposed for improving the viability of the cells thus set in the alginate matrix (A method for the large-scale cultivation of animal cells wherein animal cells are embedded in a collagen gel which is covered by a protective coating. The protective coating supports and protects the collagen matrix. Junpei Enami, Naohito Kondo, Toshikazu Takano, Kaneo Suzuki., U.S. Pat. No. 5,264,359., 1989).

A second problem lies in the lack of room inside the gel for growing the cells. Different strategies were therefore tested as to the formation of capsules (Encapsulation of biological material, Franklin Lim, U.S. Pat. No. 4,352,883, 1982, Tissue culture and production in permeable gels., Elizabeth Maureen Frye, Mark Maurice Lynch, John Paul Vasington., EP 0 185 701, 1984).

Patent application WO 2010/063937 describes a method for preparing capsules having a liquid core and a gelled shell of a small thickness totally encapsulating the core. These capsules are formed by co-extrusion of drops at the outlet of a jacket. Said capsules are described as being able to contain cells. Nevertheless, even if the method seems to be adapted for cell encapsulation, nothing indicates that the capsules give the possibility of supporting cell growth when encapsulated cells are cultivated, neither especially the amplification and differentiation of cells with hematopoietic potential, in particular hematopoietic stem cells or erythroid progenitor cells.

The inventors have demonstrated that capsules containing cells with hematopoietic potential according to the invention give the possibility of solving all these problems and therefore give the possibility of contemplating for the first time an application to cell culture of cells with hematopoietic potential at a large scale.

The object of the invention is thus to improve the yield of cell cultures of cells with hematopoietic potential, for producing enucleated erythroid cells in a bioreactor. The challenge is to bring the nutriments and to remove the catabolites required for good growth of cells with hematopoietic potential.

The capsule according to the invention gives the possibility of protecting said cells from shearing or from oxygen bubbles in solution, while guaranteeing homogenous growth conditions.

With a wall porous to small molecules and with a size of less than one millimeter, diffusion is actually sufficient for guaranteeing the homogeneity in concentration inside the capsules. As the wall protects the cells, it will be possible to stir a large concentration of capsules efficiently and at a large scale in the bioreactor. The encapsulation has other advantages for cultivating cells with hematopoietic potential at a large scale: the capsules are simple to handle (by sedimentation, capture through a filter) and will for example give the possibility of easily changing the culture medium without applying stress on the cells, unlike a conventional reactor.

Thus, the present invention relates to a capsule comprising a liquid core, a gelled envelope totally encapsulating the liquid core at its periphery, the gelled shell being able to retain the liquid core when the capsule is immersed in a gas, the gelled shell comprising at least one gelled polyelectrolyte and at least one surfactant, for which the liquid core comprises at least one cell with hematopoietic potential.

By “cells with hematopoietic potential” are meant cells capable of differentiating towards one or several of the lines which are at the origin of blood cells.

More particularly, the “cells with hematopoietic potential” according to the invention are cells of the erythroid line, which may produce red corpuscles during their differentiation. The cells with hematopoietic potential according to the invention may notably stem from stem cells, in particular embryonic stem cells (ESC), adult stem cells, like hematopoietic stem cells BSC), pluripotent induced stem cells (iPS), immortalized cells, erythroid progenitor cells or erythroid precursors. Preferably, the “cells with hematopoietic potential” of the invention are human cells.

Preferably, the cells with hematopoietic potential are hematopoietic stem cells and/or erythroid progenitor cells and/or erythroid precursors.

When the cells with hematopoietic potential are hematopoietic stem cells, the latter are preferably human hematopoietic stem cells, in particular CD34+ cells which may be obtained from umbilical cord blood or by leukophoresis, from peripheral blood. They preferentially will differentiate into reticulocytes and/or erythrocytes.

Erythrocytes or hematia, are more commonly called red corpuscles.

Reticulocytes are the cells preceding the erythrocyte stage in erythropoiesis. They are quasi similar to them. Reticulocytes are young red corpuscles which still have ribosomes and mitochondria, but are without any peroxisome.

By “erythroid progenitor cell”, is meant a cell stemming from the differentiation of a hematopoietic stem cell, capable of proliferating and differentiating into a cell of the erythroid line, in particular into reticulocytes and/or erythrocytes. They are preferably human erythroid progenitor cells.

By “erythroid precursor”, is meant any cell from the cytologically identifiable erythroid line, i.e. meeting conventional criteria for identifying cell types ranging from proerythroblast to acidophilic erythroblasts. They are preferably human erythroid precursors.

Within the scope of the present invention, the liquid core of the capsule may comprise at the moment of the encapsulation an amount of cells with hematopoietic potential comprised between 1 and 10,000, preferably between 10 and 1,000 cells/capsule.

The liquid core of the capsule according to the invention consist of a liquid preferably physiologically acceptable, such as a saline solution, a buffer solution, a physiologically acceptable viscosifying agent and/or a culture medium intended for growth and differentiation of cells with hematopoietic potential, the composition of which will be detailed in the subsequent description.

The liquid core may also comprise physiologically acceptable excipients, such as thickeners, or rheology modifiers. These thickeners are for example polymers, cross-polymers, microgels, gums or proteins, including polysaccharides, celluloses, polyosides, polymers and co-polymers based on silicone, colloidal particles (silica, clays, latex . . . ).

The gelled shell of the capsules according to the invention comprises a gel containing water and at least one polyelectrolyte reactant to multivalent ions. According to the invention, the shell further contains a surfactant resulting from its manufacturing method, as described subsequently.

In particular, the capsule according to the invention is obtained from a method comprising the following steps:
a) separately conveying in a jacket a first liquid solution containing at least one cell with hematopoietic potential and of a second liquid solution containing a liquid polyelectrolyte able to be gelled;
b) forming, at the outlet of the jacket, a series of drops, each drop comprising a central core formed with said first solution and a peripheral film formed with said second solution and totally covering the central core;
c) immersing each drop in a gelling solution containing a reagent able to react with the polyelectrolyte of the film so as to have it pass from a liquid state to a gelled state and form the gelled shell, the central core forming the liquid core;
d) recovering the formed capsules;
   the second solution containing at least one surfactant before its contact with the first solution.

According to one of the aspects of this method, the flow rate ratio of the first solution to the flow rate of the second solution at the outlet of the jacket is comprised between 1 and 200, advantageously between 10 and 200, the gelled shell having a thickness comprised between 0.1% and 10%, advantageously between 0.1% and 2% of the diameter of the capsule, after recovering the formed capsules.

According to another aspect of the method, the first physiologically acceptable liquid solution comprises a saline solution, a buffer solution, a physiologically acceptable viscosifying solution, a physiologically acceptable excipient, advantageously a thickener or rheology modifier, and/or of the culture medium.

According to another aspect of the method, the drops formed by co-extrusion in the jacket fall by gravity through a volume of air in the gelling solution.

Within the scope of the present description, by “surfactant” is meant an amphiphilic molecule having two portions with different polarity, one is lipophilic and apolar, the other hydrophilic and polar. A surfactant may be of the ionic type (cationic or anionic), zwitterionic or non-ionic type.

The surfactant is advantageously an anionic surfactant, a non-ionic surfactant, a cationic surfactant or a mixture thereof. The molecular mass of the surfactant is comprised between 150 g/mol and 10,000 g/mol, advantageously between 250 g/mol and 1,500 g/mol.

In the case when the surfactant is an anionic surfactant, it is for example selected from among an alkylsulfate, an alkyl sulfonate, an alkylarylsulfonate, an alkaline alkylphosphate, a dialkylsulfosuccinate, an earth-alkaline salt of either saturated fatty acids or not. These surfactants advantageously have at least one hydrophobic hydrocarbon chain having a number of carbon atoms of more than 5, or even 10 and at least one hydrophilic anionic group, such as a sulfate, a sulfonate or a carboxylate bound to one end of the hydrophobic chain.

In the case when the surfactant is a cationic surfactant, it is for example selected from among a halide salt of alkylpyridium or alkylammonium like n-ethyldodecylammonium chloride or bromide, cetylammonium chloride or bromide (CTAB). These surfactants advantageously have at least one hydrophobic hydrocarbon chain having a number of carbon atoms of more than 5, or even 10 and at least one hydrophilic cationic group, such as a quaternary ammonium cation.

In the case when the surfactant is a non-ionic surfactant, it is for example selected from polyoxyethylene and/or polyoxypropylene derivatives of fatty alcohols, of fatty acids, or alkylphenols, arylphenols, or from among alkyl glucosides, polysorbates, cocamides.

In particular, the surfactant will be selected from the following list: an alkylsulfate, an alkyl sulfonate, an alkylarylsulfonate, an alkaline alkylphosphate, a dialkylsulfosuccinate, an earth-alkaline salt of saturated fatty acids or not, a halide salt of alkylpyridium or alkylammonium like n-ethyldodecylammonium chloride or bromide, cetylammonium chloride or bromide, polyoxyethylene and/or polyoxypropylene derivatives of fatty alcohols, of fatty acids or alkylphenols, or from among arylphenols, alkyl glucosides, polysorbates, cocamides or mixtures thereof.

More particularly, the total mass percentage of surfactant in the second solution will be greater than 0.01% and is advantageously comprised between 0.01% and 0.5% by mass.

By “polyelectrolyte reactive to polyvalent ions”, is meant in the sense of the present invention, a polyelectrolyte which may pass from a liquid state in an aqueous solution to a gelled state under the effect of contact with a gelling solution containing multivalent ions such as ions of an earth-alkaline metal for example selected from among calcium ions, barium ions, magnesium ions.

In the liquid state, the individual polyelectrolyte chains are substantially free to flow relatively to each other. An aqueous solution of 2% by mass of polyelectrolyte then has a purely viscous behavior at the characteristic shearing gradients of the shaping method.

The viscosity of this solution with zero shearing is between 50 mPa·s and 10,000 mPa·s, advantageously between 3,000 mPa·s and 7,000 mPa·s.

The polyelectrolyte individual chains in the liquid state advantageously have a molar mass of more than 65,000 g/mol.

In the gelled state, the polyelectrolyte individual chains form, with the multivalent ions, a coherent three-dimensional network which retains the liquid core and prevents its flow. The individual chains are retained relatively to each other and cannot freely flow relatively to each other. In this state, the viscosity of the formed gel is infinite. Further, the gel has a stress threshold to flowing, this stress threshold is greater than 0.05 Pa. The gel also has a non-zero elastic modulus and greater than 35 kPa.

The three-dimensional gel of polyelectrolyte contained in the shell confines the water and the surfactant.

The polyelectrolyte is preferably a harmless biocompatible polymer for the human body. For example it is produced biologically.

Advantageously, it is selected from among polysaccharides, synthetic polyelectrolytes based on acrylates (sodium polyacrylate, lithium polyacrylate, potassium or ammonium polyacrylate, or polyacrylamide), on synthetic polyelectrolytes based on sulfonates (sodium poly(styrene sulfonate) for example). More particularly, the polyelectrolyte is selected from an earth-alkaline alginate, such as a sodium alginate or a potassium alginate, a gellan or a pectin.

The alginates are produced from brown algae called <<luminaries>> designated by the term of <<seaweed>>.

Such alginates advantageously have an α-L-glucuronate content greater than about 50%, preferably greater than 55%, or even greater than 60%.

In particular, said or each polyelectrolyte will be a polyelectrolyte reactive to multivalent ions, notably a polysaccharide reactive to multivalent ions such as an alkaline alginate, a gellan or a pectin, preferably an alkaline alginate advantageously having a block α-L-glucoronate content greater than 50%, notably greater than 55%.

More particularly, the mass polyelectrolyte content in the second solution may be less than 5% by mass and is advantageously comprised between 0.5 and 3% by mass.

According to an aspect of the present invention, the capsule may further comprise an intermediate shell totally encapsulating at its periphery the liquid core, said intermediate shell being itself totally encapsulated at its periphery by the gelled shell.

This intermediate liquid shell will be formed with an intermediate composition comprising a buffer or cell culture medium, and/or a viscosifying agent. In particular, the viscosifying agent will be a waters-soluble polymer, such as PEG, dextran or further of the alginate in a more diluted solution than in the outer shell.

The intermediate shell is in contact with the core and the outer shell and maintains the core out of contact of the outer shell.

The intermediate phase is useful for stabilizing the capsule during its formation, for example in the case when the liquid core contains calcium which may induce too early the gelling of the external phase. Indeed, depending on their composition, the cell culture media may interfere with polymerization. Thus it gives the possibility of separating the liquid core from the outer phase to be gelled. It also gives the possibility of protecting the liquid core containing the cells during the formation of the drops, of the alginate of the external layer which is not yet polymerized.

In particular, as the liquid core and the intermediate phase are all both liquid, they mix together in the long run in order to form the liquid core of the capsule.

The presence of such an intermediate shell is notably described in the scientific article “Formation of liquid-core capsules having a thin hydrogel membrane: liquid pearls”, Bremond et al, Soft Matter, 2010, 2484-2488.

Production of the Drops

The production of the drops according to the method according to the invention mentioned earlier is carried out by conveying separately in a jacket a first liquid solution containing the cell(s) with hematopoietic potential and of a second liquid solution containing a liquid polyelectrolyte able to gel and at least one surfactant, as described in WO2010/063937.

In the case of the additional presence of an intermediate shell, the separate conveyance is carried out in a triple shell, with a third solution comprising the intermediate solution.

At the outlet of the double (or triple) shell, the different flows come into contact and then form a multi-component drop, according to a hydrodynamic model a so called <<dripping>>mode (drop wise, notably described in WO 2010/063937) or a so called <<jetting>>mode (with a jet instability, notably described in FR 2012/2964017), depending on the size of the desired capsules.

The first flow forms the liquid core and the second flow forms the liquid external shell. In the case of the presence of the intermediate shell, the second flow forms the liquid intermediate shell and the third flow the liquid external shell.

According to the production mode, each multi-component drop is detached from the double (or triple) shell and falls into a volume of air, before being immersed into a gelling solution containing a reagent able to gel the polyelectrolyte of the liquid external shell, in order to form the gelled external shell of the capsules according to the invention.

According to certain alternatives, the multi-component drops may comprise additional layers between the external shell and the liquid core, other than the intermediate shell. This type of drop may be prepared by conveying separately multiple compositions in devices with multiple shell.

Gelling Step

When the multi-component drop comes into contact with the gelling solution, the reagent able to gel the polyelectrolyte present in the gelling solution then forms bonds with the different polyelectrolyte chains present in the liquid external shell, then passing to the gelled state, thereby causing gelling of the liquid external shell.

Without intending to be bound to a particular theory, during the passing to the gelled state of the polyelectrolyte, the individual polyelectrolyte chains present in the liquid external shell connect together in order to form a cross-linked network, also called a hydrogel.

Within the scope of the present description, the polyelectrolyte present in the gelled external shell is in the gelled state and is also called a polyelectrolyte in the gelled state or further a gelled polyelectrolyte.

A gelled external shell, able to retain the assembly formed by the core or the core and the intermediate shell, is thereby formed. This gelled external shell has a specific mechanical strength, i.e. it is capable of retaining the liquid core and, in the case of the presence of an intermediate shell, of totally surrounding the intermediate shell. This has the effect of maintaining the internal structure of the liquid core and if necessary of the intermediate shell.

The capsules according to the invention dwell in the gelling solution for a period during which the external shell is completely gelled, preferably without exceeding 30 minutes, still more preferentially without exceeding 5 minutes.

Next it is optionally possible to remove the gelling solution and the gelled capsules may then optionally be collected and immersed in an aqueous rinsing solution, generally essentially consisting of water, in particular physiological water, in order to rinse the formed gelled capsules. This rinsing step allows extraction from the gelled external shell, a possible excess of the reagent able to gel of the gelling solution, and all or part of the surfactant (or other species) initially contained in the second liquid solution.

The presence of a surfactant in the second liquid solution gives the possibility of improving the formation and the gelling of the multi-component drops according to the method as described earlier.

Characteristics of the Gelled Capsules

Advantageously, the capsule is of a spherical shape and has an outer diameter of less than 5 mm and notably comprised between 0.3 mm and 3 mm.

Preferably, the gelled external shell of the capsules according to the invention have a thickness comprised from 10 μm to 500 μm, preferably from 20 μm to 200 μm, and advantageously from 50 μm to 100 μm.

The fineness of the thickness of the gelled external shell generally gives the possibility of making this external shell transparent.

The capsules according to the invention generally have a volume ratio between the core and the whole of the intermediate and external shells greater than 2, and preferably less than 50.

According to a particular embodiment, the capsules according to the invention generally have a volume ratio between the core and the whole of the intermediate and external shells comprised between 5 and 10.

The invention also relates to the use of at least one capsule as described within the scope of the present invention, for ex vivo production of enucleated erythroid cells, in particular of reticulocytes and/or erythrocytes.

It also relates to an ex vivo method for producing enucleated erythroid cells comprising the culture of cells with a hematopoietic potential contained in at least one capsule as defined within the scope of the present invention, under conditions allowing production of enucleated erythroid cells.

Particularly, the enucleated erythroid cells produced according to the method of the present invention are reticulocytes and/or erythrocytes.

In particular, the cells with hematopoietic potential used within the scope of the method of the invention are hematopoietic stem cells and/or erythroid progenitor cells and/or erythroid precursors, more particularly human erythroid precursors.

Thus, the cells with hematopoietic potential may be cultivated within the scope of the present invention in a culture medium comprising:
a) insulin at a concentration comprised between 1 and 50 μg/ml;
b) transferrin at a concentration comprised between 100 and 2,000 μg/ml; and
c) plasma or serum at a concentration comprised between 1 and 30%.

By “culture medium”, is meant any medium, in particular any liquid medium which may support the growth of cells with hematopoietic potential, in particular hematopoietic stem cells, erythroid progenitor cells and erythroid precursors, more particularly human erythroid precursors, and allowing the production of enucleated erythroid cells, in particular reticulocytes and/or erythrocytes.

The insulin of the culture medium according to the present invention is in particular human recombinant insulin. Its concentration is preferentially comprised between 5 and 20 μg/ml, more preferentially between 8 and 12 μg/ml, and even more preferentially 10 μg/ml.

The transferrin of the culture medium according to the invention in particular is human transferrin. More particularly, the transferrin is saturated with iron. Its concentration is preferentially comprised between 200 and 1,000 μg/ml, more preferentially between 300 and 500 μg/ml, and even more preferentially of 330 or 450 μg/ml. The transferrin may also appear in a recombinant form.

The plasma or the serum of the culture medium according to the invention are in particular human. Their concentration is preferentially comprised between 1 and 20%, more preferentially between 4 and 12%, and still more preferentially of 5 or 10%.

According to an aspect of the present invention, the culture medium also comprises heparin, in particular at a concentration comprised between 0.5 and 5 UI/ml, preferentially between 1.5 and 3.5 UI/ml, and still more preferentially 2 UI/ml. In particular, the culture medium according to the present invention comprises heparin when the culture medium also comprises plasma.

The culture medium may also comprise ‘EPO and/or SCF and/or IL-3 and/or hydrocortisone.

EPO (erythropoietin) of the culture medium according to the invention is in particular recombinant human EPO. Its concentration is preferentially comprised between 0.5 and 20 UI/ml, more preferentially between 2.5 and 3.5 UI/ml, and still more preferentially 3 UI/ml.

SCF (Stem Cell Factor) of the culture medium according to the invention is in particular recombinant human SCF. Its concentration is preferentially comprised between 50 and 200 ng/ml, more preferentially between 80 and 120 ng/ml, and still more preferentially 100 ng/ml.

IL-3 (interleukin 3) of the culture medium according to the invention is in particular recombinant human IL-3. Its concentration is preferentially comprised between 1 and 30 ng/ml, more preferentially between 4 and 6 ng/ml, and still more preferentially 5 ng/ml.

Hydrocortisone which is optionally added into the culture medium, has according to the invention preferentially a concentration comprised between 5·10⁷ and 5·10⁻⁶ M, and more preferentially 5·10⁻⁶ M.

According to an aspect of the present invention, the culture medium may comprise at least one of the following compounds: TPO, FLT3, BMP4, VEGF-A165 and IL-6.

TPO (thrombopoietin) of the culture medium according to the invention is in particular recombinant human TPO. Its concentration is preferentially comprised between 20 and 200 ng/ml, more preferentially between 80 and 120 ng/ml, and still more preferentially 100 ng/ml.

FLT3 (FMS-like tyrosine kinase 3 ligand) of the culture medium according to the invention is in particular recombinant human FLT3. Its concentration is preferentially comprised between 20 and 200 ng/ml, more preferentially between 80 and 120 ng/ml, and still more preferentially 100 ng/ml.

BMP4 (Bone Morphogenic Protein 4) of the culture medium according to the invention is in particular recombinant human BMP4. Its concentration is preferentially comprised between 1 and 20 ng/ml, more preferentially between 8 and 12 ng/ml, and still more preferentially 10 ng/ml.

VEGF-A165 (Vascular Endothelial Growth Factor A165) of the culture medium according to the invention is in particular recombinant human VEGF-A165. Its concentration is preferentially comprised between 1 and 20 ng/ml, more preferentially between 4 and 6 ng/ml, and still more preferentially 5 ng/ml.

L'IL-6 (interleukin 6) of the culture medium according to the invention is in particular recombinant human IL-6. Its concentration is preferentially comprised between 1 and 20 ng/ml, more preferentially between 4 and 6 ng/ml, and still more preferentially 5 ng/ml.

Within the scope of the present invention, the culture medium comprises a basic culture medium, the latter having the characteristic of being capable of supporting growth of cells with hematopoietic potential, in particular hematopoietic stem cells, erythroid progenitor cells and/or erythroid precursors, more particularly human erythroid precursors, and of allowing the production of enucleated erythroid cells, in particular reticulocytes and/or erythrocytes. This type of basic culture medium is well known to one skilled in the art. For example mention may be made of the modified Iscove Dulbecco's medium (IMDM), completed with glutamine or a peptide containing glutamine.

Thus, the culture medium according to the present invention also preferentially comprises modified Iscove Dulbecco's medium, completed with glutamine or a peptide containing glutamine.

In particular, the cells with hematopoietic potential are cultivated in a medium comprising:

during a first step from 5 to 9 days, in particular 7 days:
insulin at a concentration comprised between 8 and 12 μg/ml;
transferrin at a concentration comprised between 300 and 350 μg/ml;
plasma at a concentration comprised between 3 and 7%;
heparin at a concentration comprised between 1.5 and 2.5 IU/ml;
optionally hydrocortisone at a concentration comprised between 5·10⁷ and 5·10⁻⁶ M;
SCF at a concentration comprised between 80 and 120 ng/ml;
IL-3 at a concentration comprised between 4 and 6 ng/ml; and
EPO at a concentration comprised between 2.5 and 3.5 IU/ml;

and then during a second step from 0 to 5 days, in particular from 3 to 4 days:
insulin at a concentration comprised between 8 and 12 μg/ml;
transferrin at a concentration comprised between 300 and 350 μg/ml;
plasma at a concentration comprised between 3 and 7%;
heparin at a concentration comprised between 1.5 and 2.5 IU/ml;
optionally hydrocortisone at a concentration comprised between 5·10⁷ and 5·10⁻⁶ M;
SCF at a concentration comprised between 80 and 120 ng/ml; and
EPO at a concentration comprised between 2.5 and 3.5 IU/ml;

and in a third step from 6 to 10 days, in particular up to 18 or 21 days from the beginning of the first step:
insulin at a concentration comprised between 8 and 12 μg/ml;
transferrin at a concentration comprised between 300 and 350 μg/ml;
plasma at a concentration comprised between 3 and 7%;
heparin at a concentration comprised between 1.5 and 2.5 IU/ml; and
EPO at a concentration comprised between 2.5 and 3.5 IU/ml.

Alternatively, the cells with hematopoietic potential are cultivated in a medium comprising:

during a first step from 6 to 8 days, in particular 7 days:
insulin at a concentration comprised between 8 and 12 μg/ml;
transferrin at a concentration comprised between 300 and 350 μg/ml;
plasma at a concentration comprised between 1% and 7%;
heparin at a concentration comprised between 1.5 and 2.5 IU/ml;
SCF at a concentration comprised between 80 and 120 ng/ml;
IL-3 at a concentration comprised between 5 and 30 ng/ml;
TPO at a concentration comprised between 80 and 120 ng/ml; and
FLT3 at a concentration comprised between 30 and 60 ng/ml;

and then during a second step from 10 to 16 days, in particular 14 days:
insulin at a concentration comprised between 8 and 12 μg/ml;
transferrin at a concentration comprised between 300 and 350 μg/ml;
plasma at a concentration comprised between 1% and 7%;
heparin at a concentration comprised between 1.5 and 2.5 IU/ml;
optionally hydrocortisone at a concentration comprised between 5·10⁷ and 5·10⁻⁶ M;
SCF at a concentration comprised between 80 and 120 ng/ml;
EPO at a concentration comprised between 2.5 and 3.5 IU/ml; and
IL-3 at a concentration comprised between 4 and 6 ng/ml;

and in a third step from 6 to 12 days, in particular up to 28 or 32 days from the beginning of the first step:
insulin at a concentration comprised between 8 and 12 μg/ml;
transferrin at a concentration comprised between 300 and 350 μg/ml;
plasma at a concentration comprised between 1% and 7%;
heparin at a concentration comprised between 1.5 and 2.5 IU/ml; and
EPO at a concentration comprised between 2.5 and 3.5 IU/ml.

Alternatively, the cells with hematopoietic potential are cultivated in a medium comprising:

during a first step from 5 to 25 days, in particular 20 days:
insulin at a concentration comprised between 8 and 12 μg/ml;
transferrin at a concentration comprised between 425 and 475 μg/ml;
plasma at a concentration comprised between 3 and 7%;
heparin at a concentration comprised between 1.5 and 2.5 IU/ml;
SCF at a concentration comprised between 80 and 120 ng/ml;
TPO at a concentration comprised between 80 and 120 ng/ml;
FLT3 at a concentration comprised between 80 and 120 ng/ml;
BPM4 at a concentration comprised between 8 and 12 ng/ml;
VEGF-A165 at a concentration comprised between 4 and 6 ng/ml.
IL-3 at a concentration comprised between 4 and 6 ng/ml;
IL-6 at a concentration comprised between 4 and 6 ng/ml; and
EPO at a concentration comprised between 2.5 and 3.5 IU/ml;

and then during a second step from 6 to 10 days, in particular 8 days:
insulin at a concentration comprised between 8 and 12 μg/ml;
transferrin at a concentration comprised between 425 and 475 μg/ml;
plasma at a concentration comprised between 8 and 12%;
heparin at a concentration comprised between 2.5 and 3.5 IU/ml;
SCF at a concentration comprised between 80 and 120 ng/ml;
IL-3 at a concentration comprised between 4 and 6 ng/ml; and
EPO at a concentration comprised between 2.5 and 3.5 IU/ml;

and then during a third step from 2 to 4 days, in particular 3 days:
insulin at a concentration comprised between 8 and 12 μg/ml;
transferrin at a concentration comprised between 425 and 475 μg/ml;
plasma at a concentration comprised between 8 and 12%;
heparin at a concentration comprised between 2.5 and 3.5 IU/ml;
SCF at a concentration comprised between 80 and 120 ng/ml; and
EPO at a concentration comprised between 2.5 and 3.5 IU/ml;

and in a fourth step from 10 to 16 days, in particular 13 days:
insulin at a concentration comprised between 8 and 12 μg/ml;
transferrin at a concentration comprised between 425 and 475 μg/ml;
plasma at a concentration comprised between 8 and 12%;
heparin at a concentration comprised between 2.5 and 3.5 IU/ml; and
EPO at a concentration comprised between 2.5 and 3.5 IU/ml.

Within the scope of the present invention, the cells are preferably encapsulated at DO i.e. at a not very advanced differentiation stage, for example at that of hematopoietic stem cells, but may also be encapsulated at a subsequent stage, i.e. at the stage of progenitor or of erythroid precursor.

The capsules according to the invention comprising at least one cell with a hematopoietic potential, may therefore exclusively comprise not very differentiated cells like hematopoietic stem cells, only cells with a more advanced differentiation stage such as erythroid progenitor cells, erythroid precursors or a mixture of cells with hematopoietic potential at different differentiation stages.

The present invention will be illustrated in more detail by the figures and examples below which do not limit the scope thereof.

### FIGURES

FIG. 1: Principle of the preparation of the capsules:

A triple shell structure is illustrated wherein the fluid forming the core (which comprises at least one cell with hematopoietic potential) is introduced into the central shell indicated by the vertical arrow while the intermediate fluid is introduced into the intermediate shell indicated by the arrow located on the left of the triple shell structure and the fluid of the shell in the outer shell indicated by the arrow located on the right of the triple shell structure.

The thereby formed capsules fall into the gelling bath.

FIG. 2: Perspective view of the injector.

FIG. 3: Sectional view of the injector:

The injector comprises three inlets, the first inlet located on the left of the sectional view corresponding to the entry of the intermediate fluid, the second corresponding inlet to the first inlet located on the top of the corresponding sectional view at the entry of the core fluid and the third inlet corresponding to the second inlet located on top of the corresponding sectional view at the entry of the shell fluid.

FIG. 4: Encapsulation of the cells.

### EXAMPLE

### Cultivation of Encapsulated Cells with Hematopoietic Potential

### Preparation of the Cells

The cells CD34+ are isolated from placenta blood by selection with supermagnetic microbeads by using Mini-MACS columns (Miltenyi Biotech, Bergisch Glodbach, Germany) (purity greater than 94±3%).

The cells are cultivated in an IMDM medium (Iscovemodified Dulbecco's medium, Biochrom, Germany) supplemented with 2 mM of L-glutamine (Invitrogen, Cergy-Pontoise, France), 330 μg/ml of human transferrin saturated with iron, 10 μg/ml of insulin (Sigma, Saint-Quentin Fallavier, France), 2 IU/ml of heparin Choay (Sanofi, France) and 5% of plasma (solvent/detergent virus inactivated plasma (S/D)), 10⁴ cells CD34+/ml cultivated in the presence of 100 ng/ml of SCF(provided by Amgen, Thousand Oaks, Calif.), 5 ng/ml of IL-3 (R&D Systems, Abingdon, United Kingdom) and 3 IU/ml of EPO(Eprex, provided by Janssen Cilag, Issy-les-Moulineaux, France). On day 4, a volume of cell culture is diluted in four volumes of fresh medium containing SCF, IL-3 and EPO. The cultures are maintained at 37° C. with 5% of CO₂ in air. On day 8, the cells are counted and their concentration is adjusted in the encapsulation medium (IMDM, heparin, plasma, insulin, transferrin, EPO, SCF and IL-3) in order to attain the desired concentration.

The cells are encapsulated on D8.

### Procedure for Encapsulating the Cells

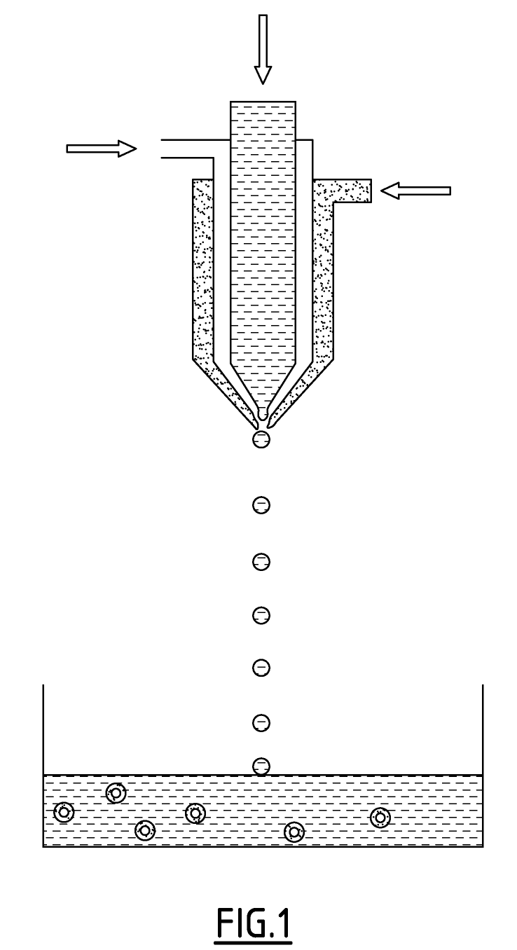

The general principle of forming capsules is indicated in FIG. 1.

The day before the encapsulation procedure, the following elements were prepared.

One litre of 1% CaCl₂ was filtered on a 0.2 μm filter in order to be used in the gelling bath. Two liters of physiological water (saline NaCl 0.9%) are prepared which will be used for rinsing the set up, i.e. the injector, the tubes, the syringes and the connectors forming the encapsulation device, as well as the capsules. One litre of milliQ water is also prepared for setting into place the set up under water. Finally, a solution of 30 ml of 2% alginate with 0.5 mM of SDS is prepared. The following elements were autoclaved: crystallizer, 10 beakers of 150 ml (for recovering the capsules and rinsing them), a clamp, microfluidic connectors (for the tube diameter 1/16 inch), tubes in Teflon (diameter 1/16 inch; 60 cm).

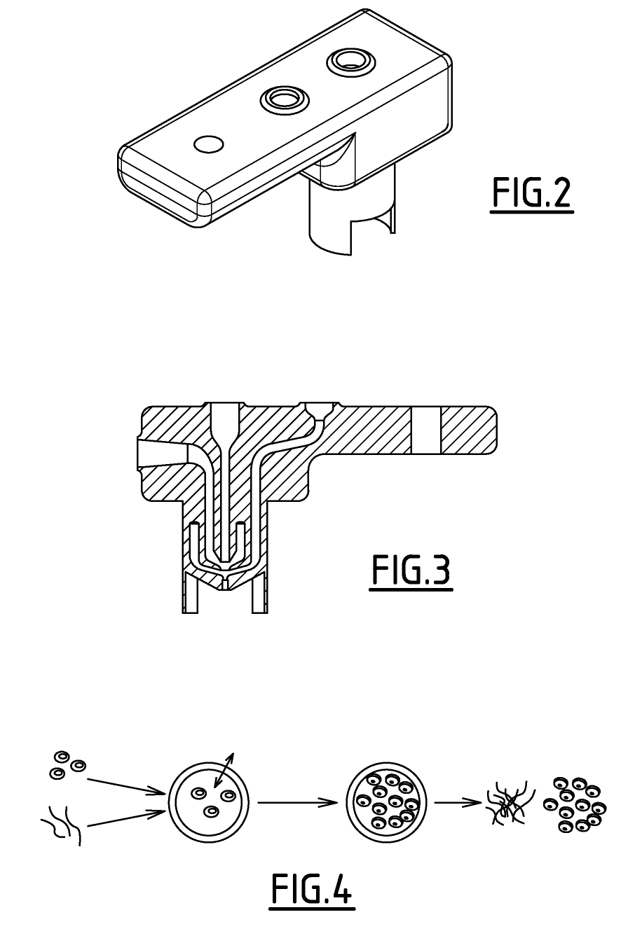

The next day, the encapsulation device is mounted according to the following operating procedure: according to the set up is washed with 70% EtOH and then with filtered milliQ water (injector XII see FIGS. 2 and 3). It comprises capillaries for which the inner diameter is of 0.78 mm and the outer diameter of 1 mm as well as 3 syringes SGE (VWR), that for the internal phase of 5 ml with stirring, those for the two other phases of 10 ml. The set-up is mounted under water so as to avoid the presence of bubbles and is then filled with 10% PBS/FCS (fetal calf serum) and left for one hour in order to avoid adhesion of the cells to the walls of the tubes or of the injector subsequently. It is then rinsed with physiological water. The circuit of the intermediate phase is washed with IMDM (Iscove's Modified Dulbecco's Medium) while the circuit of the internal phase is washed with the culture medium containing IMDM, heparin and plasma. The final set up is set into place with a 2% alginate solution for the shell, IMDM for the intermediate phase and with a cell suspension comprising between 1.25·10⁵ cells/ml and 2.5·10⁵ cells/ml in a complete culture medium containing IMDM, heparin, plasma, insulin, transferrin, EPO, SCF and IL-3 for the internal phase. The encapsulation is achieved as indicated in FIG. 4 by a drop wise encapsulation method (dripping). The flow rates used for the different internal-intermediate-shell phases are 5, 1 and 3 ml/h respectively. The time for forming the capsule is 4.9 seconds. The thereby obtained capsules have a diameter of 1.6 mm. They consist of an external phase which will be gelled in the presence of calcium, from an intermediate phase having the purpose of stabilizing the capsule during its formation, and of an internal phase containing the cells.

The gelling step is then carried out with a gelling bath containing a solution of calcium chloride and a drop of Tween 200 at 10%.

From 20 to 30 capsules are immersed into the gelling bath. The capsules containing the cells should not remain in the bath more than five minutes.

The calcium solution is then removed so as to drop the capsules and the latter are rinsed with 3×30 ml of physiological water. The physiological water is then removed while the capsules are re-suspended in 10 ml of culture medium. The whole is then transferred into a 25 cm² culture flask. In order that the capsules be properly immersed, the flasks are held vertically.

The cells are then cultivated in a fresh medium in the presence of EPO until D19. The cultures are maintained at 37° C. with 5% of CO₂ in the air.

### Results

The results are indicated in Table 1 below.

TABLE 1

| No. of | | | | |
|---|---|---|---|---|
| cells/capsule | | | Mortality | Final |
| [volume | Multiplication | | level | volume |
| fraction of | rate from | Enucleation | (Trypan | fraction |
| the cells] | D8 to D19 | level | Blue) | (D19) |
| 850 [0.03%] | 705 | 42% | 8% | 2% |
| 850 [0.03%] | 579 | 62% | 8% | 2% |
| 1650 [0.06%] | 352 | 47% | 14% | 2.5% |
| 1650 [0.06%] | 354 | 61% | 15% | 2.5% |
| Conventional | 585 | 70% | 10% | 0.1% |
| culture (in a | | | | |
| flask) [0.01%] | | | | |

The encapsulated cells have a red color, thereby betraying the presence of hemoglobin.

The encapsulated cells are observed after dissolving the capsule and May-GrünwaldGiemsa staining.

The presence of mitotic cells, with very few apoptotic cells and very few vacuolar cells is observed.

A 2% volume fraction of cells is obtained in the capsules after three weeks of culture, and a viability equivalent to the one measured in a static culture.

Thus, the capsules containing cells with hematopoietic potential according to the invention give the possibility of contemplating for the first time an application to the cell culture of cells with hematopoietic potential at a large scale.

1. A capsule comprising a liquid core, a gelled shell totally encapsulating the liquid core at its periphery, the gelled shell being able to retain the liquid core when the capsule is immersed in a gas, the gelled shell comprising at least one gelled polyelectrolyte and at least one surfactant, characterized in that the liquid core comprises at least one cell with hematopoietic potential.

2. The capsule according to claim 1, characterized in that it is obtained by applying a method comprising the following steps:
a) separately conveying in a jacket a first physiologically acceptable liquid solution containing at least one cell with hematopoietic potential and of a second liquid solution containing a liquid polyelectrolyte able to be gelled;
b) forming, at the outlet of the jacket, a series of drops, each drop comprising a central core formed with said first solution and a peripheral film formed with said second solution and totally covering the central core;
c) immersing each drop into a gelling solution containing a reagent able to react with the polyelectrolyte of the film so as to have it pass from a liquid state to a gelled state and forming the gelled shell, the central core forming the liquid core;
d) recovering the formed capsules;
the second solution containing at least one surfactant before its contact with the first solution.

3. The capsule according to claim 2, characterized in that the ratio of the flow rate of the first solution to the flow rate of the second solution at the outlet of the jacket is comprised between 1 and 200, advantageously between 10 and 200, the gelled shell having a thickness comprised between 0.1% and 10%, advantageously between 0.1% and 2% of the diameter of the capsule, after recovering the formed capsules.

4. The capsule according to claim 2, characterized in that the drops formed by co-extrusion in the jacket fall by gravity through a volume of air in the gelling solution.

5. The capsule according to claim 2, characterized in that the first physiologically acceptable liquid solution comprises a saline solution, a buffer solution, a physiologically acceptable viscosifying solution, a physiologically acceptable excipient, advantageously a thickener or a rheology modifier, and/or some culture medium.

6. The capsule according to claim 1, characterized in that it further comprises an intermediate shell totally encapsulating at its periphery the liquid core, said intermediate shell being itself encapsulated totally at its periphery by the gelled shell.

7. The capsule according to any of claim 1, characterized in that said at least one cell with hematopoietic potential is a hematopoietic stem cell and/or an erythroid progenitor cell and/or an erythroid precursor.

8. The capsule according to any of claim 1, characterized in that said at least one cell with hematopoietic potential or hematopoietic stem cell or erythroid progenitor cell or erythroid precursor is a human cell or precursor.

9. (canceled)

10. (canceled)

11. An ex vivo method for producing enucleated erythroid cells comprising the culture of cells with hematopoietic potential contained in at least one capsule as defined in claim 1, under conditions allowing the production of enucleated erythroid cells.

12. The method according to claim 9, characterized in that the cells with hematopoietic potential are cultivated in a culture medium comprising:
e) insulin at a concentration comprised between 1 and 50 μg/ml;
f) transferrin at a concentration comprised between 100 and 2,000 μg/ml; and
g) plasma or serum at a concentration comprised between 1 and 30%.

13. The method according to claim 10, characterized in that the culture medium also comprises EPO and/or SCF and/or IL-3 and/or hydrocortisone.

14. The method according to claim 10, characterized in that the culture medium also comprises at least one of the following compounds: TPO, FLT3, BMP4, VEGF-A 165 and IL-6.

15. The method according to claim 10, characterized in that the culture medium also comprises Iscove Dulbecco modified medium, completed with glutamine or a peptide containing glutamine.

16. The method according to claim 9, characterized in that the enucleated erythroid cells are reticulocytes and/or erythrocytes.

17. The method according to claim 9, characterized in that the cells with hematopoietic potential are hematopoietic stem cells and/or erythroid progenitor cells and/or erythroid precursors.
